(19) United States
(12) Patent Application Publication
(10) Pub. No.: US 2026/0124018 A1
(43) Pub. Date: May 7, 2026

(54) ASYMMETRIC MARKER FOR DEPTH-SURFACE IMAGING

(71) Applicant: DERMUS KFT, Budapest (HU)

(72) Inventors: Miklós GYÖNGY, Budapest (HU); Krisztián FÜZESI, Budapest (HU); Gergely CSÁNY, Budapest (HU); Gergo SZIKSZAY-MOLNÁR, Budapest (HU)

(73) Assignee: DERMUS KFT, Budapest (HU)

(21) Appl. No.: 19/380,575

(22) Filed: Nov. 5, 2025

Related U.S. Application Data
(63) Continuation-in-part of application No. 18/561,875, now abandoned.

(30) Foreign Application Priority Data
May 20, 2021 (HU) P2100200

(51) Int. Cl. A61B 90/00

(52) U.S. Cl. CPC A61B 90/39; A61B 2090/3925; A61B 2090/3937; A61B 2090/3991

(57) ABSTRACT

A planar surface marker for use with a depth surface imaging device has an asymmetric 2D shape and defines an inner space or opening sized to encompass a surface of interest. The marker is made of material that attenuates a portion of sonic energy and projects a shadow of reduced sonic energy when subjected to an ultrasound beam.

### CROSS-REFERENCE TO RELATED APPLICATIONS

This application is a continuation-in-part of U.S. application Ser. No. 18/561,875, filed Nov. 17, 2023, which in turn is a National Stage of PCT/HU2022/050026, filed Mar. 28, 2022, and claims priority to Hungarian Application No. HU P2100200, filed May 20, 2021. The entire disclosure of the aforementioned applications is incorporated herein by reference in their entirety.

### TECHNICAL FIELD

The present invention relates to a marker for depth-surface imaging, which, placed around the tissue object to be inspected, provides proper reference points for the combined registration and alignment of captured 2D images, and provides a fixed coordinate system, in which the captured 2D images can be aligned to each other with great precision.

### BACKGROUND ART

Surgeons often need preliminary information on the tissue structure under the area affecting a surgical operation. There are imaging modalities like ultrasound imaging that non-invasively reveal the internal structure of the tissue. Nevertheless, in current practice the user capturing the ultrasound images is not able to position the ultrasound images relative to the surface, since the ultrasound transceiver head covers the inspected area; thus, the exact location of the inspection is not known.

There are solutions where the user can also gain information after removing the ultrasound transducer concerning the location where the ultrasound images were taken. Nevertheless, according to current scientific knowledge, there is no such solution that provides sufficiently exact and unambiguous location information, and does not necessitate permanent marking of the tissue (e.g. with a pen); that further, makes it possible to view the registered images later, and which is able to position several ultrasound images according to the surface coordinate system.

The above needs originate from the fact that, when planning the operation, it is necessary to achieve a proper localisation precision (typically under one mm), and unambiguous location (when estimating the localisation, there must not be several possible solutions from different places). It is also a requirement that the ultrasound image records can be taken before the operation, even during a timely separate session. The diagnostics and the surgical operation are often performed separately, in most cases by different persons. The fact that the registered image records can be viewed at a later date also provides the advantage for the physician and the patient that they can follow the pathological lesion and the progress of its treatment. By being able to position several ultrasound images according to the surface coordinate system, it is possible to create a partial or full volumetric (3D) exploration, enabling the surgeon to remove all tissues to be removed, without harming any tissues that must be avoided.

Medical ultrasound examination applies, high frequency, high bandwidth sound waves (ultrasound) in the megahertz frequency range, which are reflected by the tissue to a different extent, which can be used to obtain images. Most people associate ultrasound with images of an embryo in a pregnant woman, although the scope of ultrasound examination is much broader than this. It is also used for imaging of abdominal organs, the heart, the breasts, the muscles, the tendons, vasculature (arteries and veins), and the skin. Due to speckle noise inherently present in the imaging, it is often less suitable for the examination of fine anatomic details than for example CT or MRI; but still it has several advantages, due to which it is an ideal tool in many situations, especially when the functioning of moving structures has to be examined in real time. Another great advantage is that it does not emit ionised radiation. If acoustic emission is properly chosen, no possible negative impacts are known in connection with its application, thus, this method seems fairly safe. Also, imaging is relatively cheap, and easy to implement. The real time images obtained can be used for controlled fluid drainage and tissue sampling. Doppler ultrasound examination makes it possible to assess arterial and venous flow.

Recently, by the development of technology, it is possible to create three-dimensional images by CT, MRI and ultrasound software for physicians. Traditionally, CT and MRI scans would only be able to produce two-dimensional static output. To achieve three-dimensional records, a very large number of scans must be performed, and these must be combined with certain computerised operations, to be able to create a three-dimensional model which can already be manipulated by the physician. Three-dimensional ultrasound images are also created in a very similar manner.

For acoustic imaging a transducer unit is required, which emits a sound wave, and converts the sound wave received as response to this to a signal that can be recorded. There are single element and multiple element transducers. In the transducers, typically one or more piezoelectric elements are used. As the result of electric excitation, each element creates an acoustic wave, and converts the reflected acoustic wave to an electric signal. In the case of several elements, the relative amplitude and timing of excitations, and when summing up the received signals, the relative weights and delays make it possible to modify the acoustic beam.

The most simple way of focusing is when a single fixed beam is created due to the shape of the transducer (by geometric focusing or an acoustic lens). Nevertheless, this has the disadvantage that this beam needs to be scanned somehow to enable imaging. If the transducer is composed of several, properly arranged elements, by delayed ultrasound emission of the transducer elements and by delayed summing up of the received signals, it is possible to scan the A-lines in several directions; this is called electronic scanning. If a single element transducer is used, depth information is recorded each time along one line, i.e. 1D (one-dimensional) information is read. If the elements are situated in a line (in other words, a linear transceiver is used), imaging can be performed over a plane with an acoustic lens (with each recording, a 2D image is read). If elements are situated on a plane, practically parallel with the examined surface, it is possible to scan a full 3D (three-dimensional) volume simultaneously.

US 2016/0228090 A1 describes an ultrasound imaging system having real-time tracking and image registration, which includes a fiducial marker system containing an ultrasound transmitter structured to provide a localised ultrasound pulse at an optically observable localized spot on a body of interest. The system further includes an optical imaging system, a two-dimensional ultrasound imaging system, an optical image processing system, and an ultrasound image processing system. The ultrasound imaging system further includes a registration system configured to communicate with the optical image processing system and the ultrasound image processing system, and to receive information from the image processing system, the registration system being further configured to determine a coordinate transformation that registers the optical image with the two-dimensional ultrasound image based at least partially on information concerning the spatial locations determined for the combined ultrasound and optical fudicial marker observed in the optical image and in the two-dimensional ultrasound image. For being able to locate the ultrasound position by triangulation, several markers are required. A photoacoustic system with a pulsing laser is also required, which considerably increases the costs and complexity of the construction.

WO 2017/196496 A1 describes a radiotherapy system including a radiotherapy component, a structural imaging component, a functional imaging component, and a workstation coupled to the radiotherapy component, the structural imaging component, and the functional imaging component. The workstation includes a processor, which combines the structural imaging data and functional imaging data to produce a fused model for at least a portion of the surface of interest, to generate a plan for radiotherapy treatment of the surface of interest based on the fused model, and apply, via the radiotherapy component, the radiotherapy treatment. This solution describes in general terms that by combined registration of the images acquired by the two different imaging systems, 3D imaging can be performed. Nevertheless, actual modifications in accordance with the invention are not described in the document. The application of surface markers is not covered by the document. The document only refers to the fact that the use of markers is well known in literature, thus, registration can be considered as implemented, but the difficulties posed by markers during registration of surface-depth images, and the unique marker design and the related solution detailed in this description are not mentioned. Although combined registration of the images acquired by the two imaging systems are described, no details are included in the document about the precision of 3D imaging acquired from these. The goal of the solution in accordance with our invention is explicitly good quality 3D imaging.

US 2019/374291 A1 describes a method and system for surgical image guidance. The system includes a first imaging device and an image processing system operatively connected to the first imaging device. The image processing system is configured to: receive real-time image data from the first imaging device; receive secondary image data from a second imaging device; produce enhanced composite image data by improving an alignment of physical structures in a real-time image. The image processing system is configured to operate in an unlocked mode in which the real-time image is free to move relative to the secondary image, and in a locked mode, wherein the real-time image and the secondary image are locked relative to each other to prevent relative movement therebetween. The imaging device is configured to be able to provide information to the image processing system when the image processing system is operating in the unlocked mode. It describes a solution explicitly used during surgery. Here, optical and ultrasound images are registered to each other. The solution is different from our solution in that the marker structure is different, and optical and ultrasound imaging must be performed at the same time, while in our case this is not necessarily a requirement. At least 5 markers are required for the solution, while in our case one properly designed marker is sufficient, and the registration of depth-surface images is also possible by using an appropriate beam separator design.

US 2005/234336 A1 describes methods and materials for implantable devices (markers) to permanently mark the location of biopsy or surgery for the purpose of identification. The devices are remotely delivered, preferably percutaneously. Visualisation of the markers is readily accomplished using various state-of-the-art imaging systems. Preferred visualisation is through MRI, X-ray and ultrasound. The markers function to provide evidence of the location of the lesion after the procedure is complete for reference during future examinations or procedures. The solution describes an embeddable marker; the material of which is of critical importance; only biocompatible materials can be used. It must ensure that if implemented, the location of the intervention is marked for a long period of time. Markers cannot be used for registration relative to the surface in case of individual ultrasound images, and they are also not suitable for registering the optical/surface images to other modalities. The form of the marker serves to distinguish it from natural tissues. The manufacturing of the described markers is expensive due to its critical materials, and they are not suitable for examining pathological lesions on the skin surface and the 3D structure underneath. The implantation of the markers to the appropriate location is the result of an invasive procedure, contrary to the solution in accordance with the present invention. The marker does not seem to be suitable for determining the exact spatial position of the 2D image.

US 2019/090978 A1 describes a marker delivery device, which includes a delivery catheter, a marker and a push rod. The delivery catheter is adapted to be inserted into a biopsy site. The delivery catheter includes a discharge opening. The marker includes a marker element placed in an outer carrier. The marker element contains a polymer with a plurality of microspheres configured to enhance visibility under ultrasound imaging. The marker is positioned inside the delivery catheter near the discharge opening. The push rod is positioned within the delivery catheter, and is adapted to deploy the marker from the delivery catheter into the biopsy site. Markers cannot be used for registration relative to the surface in the case of individual ultrasound images, and they are also not suitable for registering the optical/surface images to other modalities. The form of the marker serves to distinguish it from natural tissues. The manufacturing of the described markers is expensive due to the critical materials, and they are not suitable for examining pathological disturbances on the skin surface and underneath in a 3D formation. The implantation of the markers to the appropriate location is the result of an invasive procedure, contrary to the solution in accordance with the present invention.

US 2004/116802 A1 describes a medical imaging marker that includes a marking body having a shape. The marker body can comprise a mixture of materials having different imaging characteristics. The particular characteristics of the different constituent materials of the mixture can be independently controlled. The relative amounts of the materials in the mixture can be varied. The mixture can be a conventional mixture, a suspension, a composite, a glass or other mixture. The marker can be used in a plurality of imaging techniques. The medical imaging marker is different from the solution described in the present invention. The document primarily deals with a change in the material composition of the imaging marker used for the X-ray images, by decreasing the quantity of lead. In the document, the goal is not to obtain a high imaging quality, or the registration of images obtained with different imaging methods and determining their 2D and 3D position under the skin. Such information is not included in the document.

U.S. Pat. No. 5,873,827 A describes a surface marker for use in ultrasonography. The marker comprises a material which attenuates a portion of the ultrasound energy transmitted in the ultrasound field, including the marker and the tissues underlying the marker. When placed on the skin surface above a particular tissue structure and then imaged, the marker projects a shadow of reduced sonic energy into the underlying tissue structure. The shadow provides direct visual evidence that the tissue beneath the marker has been imaged. The shadow projected by the marker can also be used to locate the image of an area of particular clinical interest within the tissue structure, e.g. a tumor or cyst. In this solution, the ultrasound transducer is positioned relative to the markers using the echo shadow of the markers. In the solution in accordance with the document, several identical cross-sections may be present when using the same marker. The estimated positioning may be far from the actual position, especially when the disturbing effect of measurement errors are taken into consideration during actual measurements. The solution does not contain parallel optical imaging, neither is the combined registration of the images performed. The role of the markers is only to reduce the energy of the ultrasound so that a shadow may be created in the tissues under them, which makes it possible to determine the area within the tissue. Contrary to this, the present invention provides a marker family which, by virtue of their shapes, makes it possible to unambiguously determine the location of where the ultrasound image was taken; and, on the basis of the optical pattern on them, the optical image can also be unambiguously transformed into the marker coordinate system; thus, the images with the two modalities can be registered with each other, and displayed together.

WO 2020/047766 A1 describes a position marker with an expandable and degradable marker body that is expandable from a compact state to the expanded state. In its compact state, the marker body defines a first volume and an outer surface that has a plurality of protrusions in a compacted configuration. In its expanded state, the marker body has a second volume greater than the first volume and the plurality of protrusions are in an expanded configuration. The marker body can degrade after expanding to the expanded state. Most markers in the document are polygonal and rounded edge position markers. The document does not mention ultrasound B-mode imager positioning, and the application area refers to a surgery or biopsy operation related to some kind of a lesion. The use of the marker described is not skin surface but implanted; it can be expanded after implantation, following contact with some kind of a liquid. Their important feature is that they have two states, where the compact state is different from the extended state. The document does not describe how the position of the ultrasound image can be determined from the ultrasound image on the basis of the marker. The estimated positioning is far from the actual position, especially because of measurement errors.

WO 2012/017231 A1 describes a method for determining the extent of a structure, e.g. a non-melanoma skin cancer in or on the skin of the subject, wherein the method comprises the steps: placing an index marker on the skin adjacent to the structure; positioning an optical coherence tomography device relative to the index marker; using the optical coherence tomography device to image the structure so as to create an image of a cross-section through the skin; determining the position of an edge of the structure in the image; and translating that position of the edge in the image to a position on the skin relative to the index marker. There is a hole in the middle of the index marker, through which OCT is able to create the plurality of cross-section images, from which the 3D image is provided. The index marker does not possess special markings; therefore, it is only suitable for capturing images of the edges of the examined structure within the cross-sectional image, i.e. imaging the extent of the examined structure. The markers used in the present invention contain special markings, which enable much more precise localisation of the examined structure with several kinds of imaging methods, either acoustic imaging or optical imaging, by providing line-by-line identification of the cross-section image. The use of the marker according to the current invention and combined imaging provides a much more precise scanning and imaging capability for determining the construction of structures on the skin surface and underneath. In contrast to this, the above-mentioned marker, by its shape, is not suitable for unambiguous determination of the location of an ultrasound image, since several cross-sections of the marker are identical.

The description of the prior art documents is considered part of our description, in particular with regard to the definitions and compilations used. According to the above, several state of art documents describe hybrid imaging systems. Some of these are imaging methods used for internal mapping of the internal parts of the body, mostly blood vessels, and are based on an imaging unit in the probe to be inserted into the body, where one imaging method is used for determining the position of the probe, while the other is used for actual imaging; therefore, these are not suitable for examining lesions of the skin.

For another part of the documents reviewed above, although they could be suitable for examining lesions of the skin, they do not provide a solution for very precise determination of the 3D shape of these lesions visible on the skin and extending into the layer under the skin.

The majority of the markers described are built into the body invasively, while they do not provide sufficiently accurate reference points for very precise 3D imaging.

The goal of the invention is to eliminate the errors of the previous solutions, and to develop a marker that is able to localise very precisely the ultrasound images relative to the coordinate system of a surface image or images, so that the ultrasound images can also be registered relative to each other easily. The present invention can also be used in other technical areas where volumetric imaging looks inside a material that is not fully visible to the bare eye (by ultrasound or other image modality). Optical imaging can be replaced by another imaging modality which is only able to see the surface of the object of interest. Furthermore, if an optical image is not taken, the invention is still able to generate 3D ultrasound images from the received 2D ultrasound images.

There is still a need, therefore, for devices and methods for providing very precise, low distortion volumetric localisation for the examination of in-depth and surface formations, e.g. for certain surgical procedures. In the case of the examination of skin, invasive solutions and inserting probes are not feasible as in the case of blood vessels. The registration of 2D surface-depth images was not implemented or was difficult in many cases, and often required manual image registration; besides, low distortion imaging of the appropriate combined imaging systems could only be achieved by very expensive, complex, photoacoustic specific mirror systems. The use of the currently known markers and the related procedures does not offer a solution either, since they do not have a shape and optical pattern based on which superficial and in-depth images could be unambiguously registered to each other.

### Solution to the Problem

Neither of the former state of the art documents mention a universally usable marker as a means of completely unambiguous positioning in the case of superficial and in-depth images. The invention can help the use of a cost-effective hybrid imaging system, which enables setting up a more precise diagnosis, and/or very accurate determination of the extent of lesions under the skin surface.

### SUMMARY OF INVENTION

An asymmetric marker for depth-surface imaging device that is provided. The localisation of the 2D images, i.e. the definition of the coordinate system is aided by the marker. The marker is a planar member having an asymmetric shape with a central open space and marking the surface of interest, and eventually the 3D formation under the area of interest. Optionally, one side of the planar member can be provided with a physiologically compatible adhesive such as an acrylic-based or silicone-based pressure-sensitive adhesive used in wound dressing. Thus, optical images can freely be captured. The marker shape is completely asymmetric, and has a distinctive pattern, thus a very precise reference environment can be created for registering individual optical and acoustic images with each other.

The present asymmetric marker defines a coordinate system for combined registration of depth and surface images, either collected separately using separate devices (such as from an ultrasound imager and an optical camera), or from a single device that collects both images. The depth imager will take images from imaging planes. Such planes will intersect the asymmetric 2D shape in the form of an intersecting line that cuts out two segments from the marker. These two segments have lengths which are monotonically changing in the strict sense (either continuously increasing or decreasing) by the continuous movement of the intersecting line.

Thus, a coordinate system can be assigned for combined registration of acoustic and optical signals of a depth-surface imaging device, and if the 2D shape is a self-closing shape, an optional line can be selected so that by rotating or moving along this line it intersects sections from the 2D shape with continuously increasing or decreasing lengths at opposite ends, except at the discontinuity, where the cut-out section changes its size from maximum to minimum, if the 2D shape has an open part in any direction, an optional line can be selected so that by rotating or moving along this line it intersects sections from the 2D shape at a minimum of two sides, wherein the length of the intersected sections continuously increase or decrease.

The marker material can be waterproof paper, plastic, thin metal layer, ink layer, 3D printed plate or synthetic resin, or slightly coloured plastic. The marker can easily be placed and fixed on thin, basically flat surfaces. In the shape of the marker there is a free inner space through which the optical imaging unit captures images of the area of interest.

The marker that has a shape on which, if an imaginary line of a finite length and defined direction is drawn so that the line entirely intersects the hole in the middle of the marker and the marker segments on opposite sides of it, then the position of the line and its orientation relative to the whole marker can be unambiguously calculated from the position and dimensions of the two marker segments on the line.

An optically detectable pattern can also be printed on the surface of the marker so that the position of the optical image can also be determined according to the marker pattern coordinate system. The depth imaging system creates the image perpendicularly to the examined surface, which is allocated to the coordinate system defined by the marker pattern, i.e. it is localised according to this. The geometric arrangements of the in-depth and superficial images, including the possible image distortions, can easily be determined by a calibration measurement.

The marker that can easily be placed and fixed on thin, basically flat surfaces, containing a hole in its shape, through which the optical imaging unit takes images of the surface of interest, and has a shape on which, if an imaginary line of a finite length and defined direction is drawn so that the line entirely intersects the hole in the middle of the marker and the marker segments on opposite sides of it, then the position of the line and its orientation relative to the whole marker can be unambiguously calculated from the position and dimensions of the two marker segments on the line.

The marker, on the surface of which there is an optically detectable pattern, is printed in a way that the position of the optical image can be estimated according to a coordinate system based on the marker pattern.

The term medical imaging refers to techniques and procedures used for capturing images of the human body (or its certain parts) for clinical (medical procedures for discovering, diagnosing and examining various conditions) or scientific (including normal anatomic and physiological studies) purposes.

### BRIEF DESCRIPTION OF DRAWINGS

Hereinafter, the advantageous embodiments presenting the invention are described by figures, wherein

FIG. 1 shows the main parameters determining the shape of a marker that can be used for depth-surface imaging.

FIGS. 2A-2D show marker shapes and patterns that can be used for depth-surface imaging.

FIG. 3 shows a sample depth (ultrasound) image, wherein two sided the echo pattern and acoustic shadow of the marker can be seen with marking k and m, and the ultrasound echo pattern of the skin with marking 1.

### DETAILED DESCRIPTION OF THE INVENTION

The marker comprises a planar member having asymmetric 2D shape and defining an inner space sized to encompass the surface of interest, wherein the planar member is made of a material that attenuates a portion of sonic energy and projects a shadow of reduced sonic energy, when subjected to an ultrasound beam.

The marker can be made from a variety of materials that cause partial or full reflection at the depth imaging device, for example, but not restricted to, waterproof paper, plastic, thin metal layer, ink layer, 3D printed plate or synthetic resin. All these markers are capable of ensuring appropriate optical visibility, and also provide ultrasound detectability, concerning the latter, by a hyperecho pattern and an acoustic shadow under it. The marker material can be easily set for example in the case of an OCT imaging device to ensure high (but not full, if appropriate) reflection: for example, it can be made of coloured plastic, concerning the OCT operation wavelength.

The typical diameter of a small lesion is 5 mm; thus, the internal diameter of the marker can be 7 mm, and the outer diameter 10 mm. This ensures that if the width of the ultrasound images is at least 10 mm, the full marker width can always be captured, which is required for marker registration. It must be noted that for the success of technical implementation it is advisable but not required that the entire surface of the lesion is within the internal hole of the marker, thus, the diameter of the lesion may exceed the internal diameter of the marker. In some cases, it is worth performing an examination at certain projections—or infiltrative tumour margins—of the lesion; thus, such a projection can also be placed in the middle of the marker.

Optical imaging can be performed from any angle; it is advantageous to use an optical imaging device, for example a camera above the multimodal imaging unit. A dermatoscope image of the surface to be examined may also be taken. In both cases, the images are transformed and recorded in the computer system according to the marker coordinates.

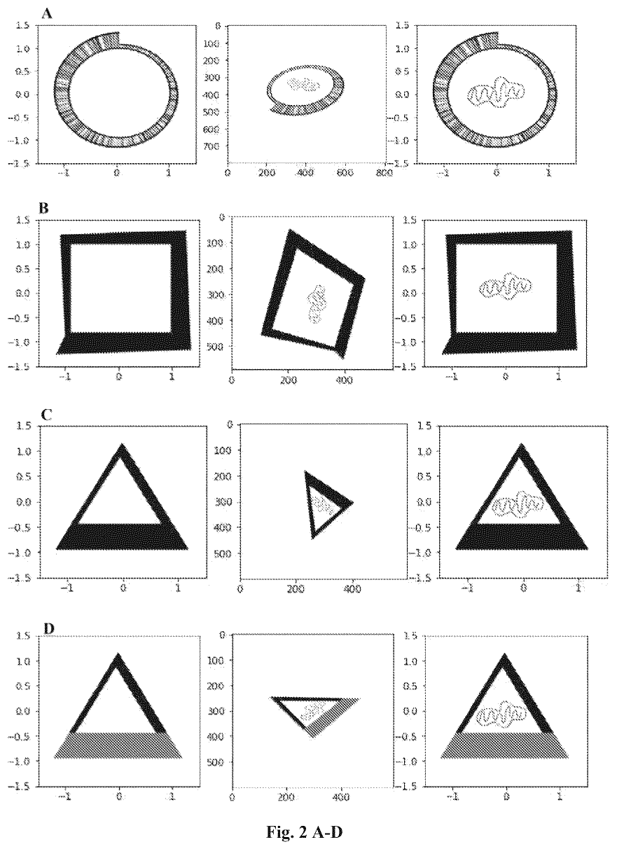

There are several solutions in literature and practice for detecting and properly transforming a marker according to the original coordinate system. Such solutions are provided by, for example, algorithms described in U.S. Pat. No. 6,711,293 B1 and US 2009/238460 A1. The marker displayed in FIG. 2A has such a pattern that these algorithms can recognise, and transform the image into the original coordinate system. Thus, the photographs are loaded into a program that transforms these images by one of the algorithms described above according to the marker coordinate system and stored, and optionally they can also be displayed. The imaging and displaying program can be a local tool running on a desktop computer, laptop, or mobile phone, or can even be a web application running on these devices. It is also conceivable that imaging is performed with a smartphone, and the program running on the smartphone transforms the image.

After this step, or simultaneously with this step, the acoustic images can also be captured, which are complemented with the acoustic image features caused by the marker. The acoustic images are transferred this way into the computerised system, defining the spatial orientation of the formations under the area to be examined.

Ultrasound imaging is widely used in medical diagnostics, and is typically performed with the so-called B-mode ultrasound imaging, which typically creates a two-dimensional in-depth image of the image plane in front of the ultrasound transceiver. In the current study, the greatest advantage of the invention concerns the two-dimensional image, therefore, we present this case, but it can also be extended to three-dimensional imaging. The applicability of the marker is not limited exclusively to the device described in the current invention; the marker can also be used for the registration of images created by any superficial and in-depth imaging device, for example, for the registration of superficial and in-depth images created by a general ultrasound B mode imaging device and the camera of a smartphone, or a general ultrasound B mode imaging device and a digital dermatoscope.

The multimodal imaging unit captures a cross-section of the marker, due to which, and as a consequence of the shape of the marker, it appears on the acoustic image as two lighter lines (with a hyperecho pattern), with an acoustic shadow behind it. The gap between the two lines is characteristic of the marker hole.

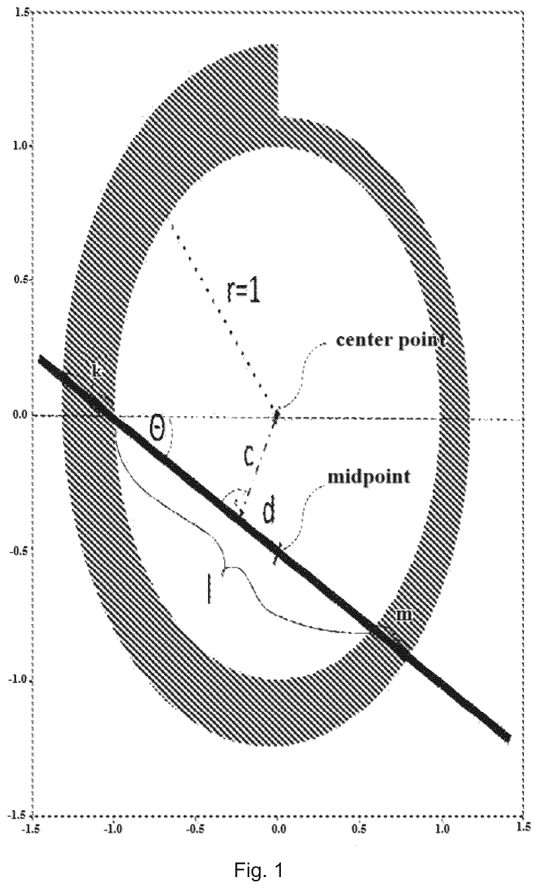

The shape of the marker is designed in such a way that the cross-section image of the marker can unambiguously identify the position of the acoustic transceiver, and through this the position of the image in the marker coordinate system. The marker coordinate system can be defined in several ways, but for presenting the invention, it is practical to describe the position line of the ultrasound transceiver (hereinafter: image line) according to the following parameters, where FIG. 1 shows a possible form of the marker:
c: the smallest distance measured from the centre of the marker (O) to the image line.
theta (θ): the angle of the image line relative to horizontal
d: the distance between the following two points:
a. the intersection point of the image line and a line starting from the centre and perpendicular to the image line
b. the centre of the image line (P)

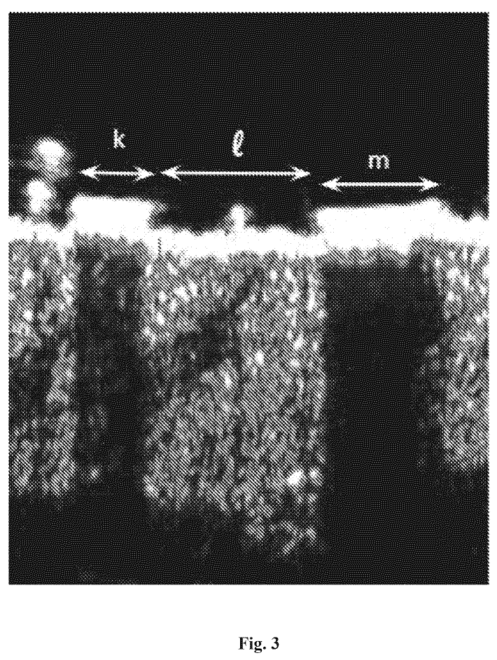

The task of the invention is to estimate these parameters from the following parameters taken from the ultrasound image (FIG. 3), where the ultrasound image detects (by detection procedures known in this field) the cross-section stripe of the two sides of the marker (also known as segments) and the hole in between:
k: the length of the left stripe
l: the length of the hole
m, the length of the right stripe
n: the distance of the hole centre from the image line centre

Thus, a subtask of the invention is to define or estimate an f([c, theta, d]) function during the inversion of which the image line parameters can be obtained; in other words:

$$g ( [ k , l , m , n ] ) = {f}^{- 1} ( [ k , l , m , n ] ) = [ c , theta , d ] ,$$

where g(.) is the inverse function of f(.).

An important characteristic of the marker is that it should provide an unambiguous solution, i.e. an unambiguous inverse of function f(.) should exist. Another characteristic is that it should be robust for noise in a way that an arbitrarily small difference should not cause a sudden change in the solution, i.e. the inverse function should be contiguous. Hereinafter, a marker that has these two characteristics will be called a suitable marker.

The suitability of the marker may cover all possible recording possibilities (c, theta, d), concerning the condition that k, l, m, and n must be measurable (the objects do not protrude out of the image partly or fully), and m>0. In such cases marker suitability can be ensured if the function f([c, theta, d]) is not contiguous in one point at most. One implementation possibility of this is when rotating the image line at an angle, k, the length of the left stripe continuously increases or decreases from a certain theta0 angle (obviously this means that when rotating from theta +180 degrees, m also continuously increases or decreases). Two implementation possibilities can be seen in FIGS. 2A and 2B.

Marker suitability may also work in a more narrow range of use. In such cases the user must be made aware in what position (typically an angle) images can be captured, and the marker can even be designed in a shape so that the ultrasound object makes it separately detectable if the image is captured out of range. Such an arrangement is presented in FIG. 2C. It may be an independent warning for the user or the program if an image captured from an angle different from the required angle causes a separate formation, if for example an extra layer causing a hyperecho is fixed to the surface to be avoided (FIG. 2D).

### Example for Use

This example describes how co-registered 2D optical and 2D ultrasound images are created using marker in accordance with the invention from an arbitrary optical camera image and the image captured by the ultrasound device.

We fitted marker onto the skin surface of interest. In the case of the lesion and marker described in FIG. 1, the pathological lesion was situated roughly in the vicinity of the centre of marker, and the pattern of marker did not cover any important parts of the lesion-similarly to FIGS. 3B-3D.

After this, we captured one or several photographs or 2D acoustic images of the skin surface of interest in a way that in every optical image the entire marker was visible, and in every 2D acoustic image the entire marker was visible, or more precisely the shadow caused by the material of marker, matching the dimensions and physical position of marker.

The trajectory of acoustic beam covered the relevant part of the skin surface of interest, and also a cross-section of marker, according to FIG. 1, thus, on the 2D acoustic image, because of the shadowing effect of marker, the sections marked with the letters k, l, and m in FIG. 1 were detectable.

The software executed on the input device mentioned in the invention can be executed on any other electronic device; it can be installed, or it can also be a web application. Thus, in addition to the device described in the invention, it is possible to capture images with any other separate optical and separate acoustic 2D imaging device, which can be processed by the software.

Since the shape of the marker was designed in a way that on the basis of the cross-section image of the marker the position of the acoustic transceiver can unambiguously be identified relative to the optical image, the images taken can also be registered with each other in the marker coordinate system.

Thus, when a marker was used, the series of superficial and in-depth images were converted by the software running on an arbitrary electronic device to a hybrid three-dimensional image, where a 2D or 3D acoustic image was also registered under the superficial optical image, depending on whether the user captured one or more 2D acoustic images. Registration of superficial and depth images was performed by detecting and measuring the optical pattern of marker and the shadow that was created during in-depth imaging caused by the material of marker, and matching the dimensions and physical position of marker, or by using an inverse function or by searching a pre-generated map or lookup table.

Finally, the images were displayed to the user by the screen of the electronic device.

1. A surface marker for the definition of a coordinate system for the combined registration of the acoustic and optical signals for the depth-surface imaging device, which comprises a planar member having asymmetric 2D shape and defining an inner space sized to encompass the surface of interest, wherein the planar member is made of a material that attenuates a portion of sonic energy and projects a shadow of reduced sonic energy, when subjected to an ultrasound beam, and the shape of the planar member is designed so that it presents a different optical and ultrasound image from every direction.

2. The marker according to claim 1, wherein the marker is a material selected from the group consisting of waterproof paper, a plastic, thin metal layer, ink layer, a 3D printed plate, synthetic resin, and coloured plastic.

3. The marker according to claim 1, provided with a physiologically compatible adhesive on one side of the planar member.

4. The marker according to claim 1, wherein an optically detectable pattern is placed or printed, with which, in addition to the shape of the marker, the position of the optical image can be more accurately determined according to the marker's coordinate system.
